SEQUENCE LISTING (39 sequences)

SEQ ID NO: 1 - DNA, 108 bp, Homo sapiens
ccagccccccttcccttccctgaccccttcttgccatcgccccagacatggggaacgcggcgaccgccaagaaaggcagcgaggtggagagcggtgagttgaaggccg

SEQ ID NO: 2 - DNA, 498 bp, Homo sapiens
agctttatatttaatgctctcattagcctatatattaatattaaaacacccaaacataaagccttttaggcagatattgcaagtttttaaaatcctcaactctagctgaaaagtgttttgctaagaaaagctcagtaatgtgctgttttatattaacaggaaacagaacagcagtagtggtttgaataccctgcaaacaggaagtttgacacatgcatagctcttagcttctgtgtaagaagttgtgagctccttctggaaacatttgcagttacattaagtaaagtgtaaatgcacatgaatggcagcttatagagaaccaccttgtaaccagtatacaggtacaactacagctcttcagaaattggaaggttttgctagccggttatttcatagacactctaaaggtactgcacatgatcagaaaacagctctggaaaatgacagccttcatttctctgaacatactgccttatgggacagatcaagtaagttttg

SEQ ID NO: 3 - DNA, 1392 bp, Homo sapiens
tgtttttaggcagagttcagtggttcgtcacaaataaatgtttcttaatttgttgtttatgactgctcgatttccagagccatgaaatcattgtttttgataattctgttcagcttcatagttgtttcttaggaagatttcctgacttacttccacatcaccaaaagtcctgcctcacatggcaactgttaaaatggcaagttcacgtgctgaagttctacttaacaaggaaccattctatagattctttgtaccattttggtacaaattttggatctctggtaatcaaaacaatctgttcaccatgctacccagtcagcccaccttgctatacaatctgtcatcttagtcctgtttcatgtgaggaattttacatttctgcaataattgccagtaacttttttgtgtattattttcttttgaataccacatggatggcatctgacactgtttgtaatgctgaatttaatggaagtttacaaataagttattctatgattctcctttaaaaatgcagatatacatatatgtatataatattattctcttccataacacagaatgtttaaatggttaacatttgtgctgcagtatagctttctggctcatgaaaaatgaaagctatcagcgatctcggcaataagattcatcgccaatagtcactagcaacagcacacagcattttaatatcagtgaggtccacagctagcagtaagagctggtgtaattgaaagacgtttaggtgcaatcattctgctgtttgctccttgccaggttcaacatgggattgtgtgagtatttgaagaaaacagcaattttttcatatctttgaaagatgtaaaaagcgtagattagtgcttaaatttaagaaatctggtaatttataatcatgtggctctaaaataaaaaggtattttatttgtctggtggattaaagctttagaaaagctacgccttggatacaagtgaaccgataattctggtctaatgttgccgtggtaacaactcatgctgatataattgagaacatcttatacatcctggttcgaacattttctccctgccattttgagttgttctagtggtatatgaaggaggctgggataactagcttgaaagaaattcagtctagttatagacatctttggcattaatctgatgtttactagtgatatctcatgctaggcagttatgctttgcttctaggggcttctctttttaaaacaaaagaaagctcttttcgttttctgtgtgctgcatgctccagtgtgtgtgtttacaccatcggttcttctccctctagagattagcataactccctttgctgttggattgttattttgagcaatatgttttggaaaggttggttttcatcatgagtggtaagtatgc

SEQ ID NO: 4 - DNA, 44 bp, Homo sapiens
cttgatcaagcacgcaaatcatcagatgcatctggtaggaaaac

SEQ ID NO: 5 - DNA, 44 bp, Homo sapiens
tggacacaagcttgctcctcttcagaaatatctggtaggcaagt

SEQ ID NO: 6 - DNA, 29 bp, Homo sapiens
gaacatgtagattcctttggtatgctcat

SEQ ID NO: 7 - DNA, 20 bp, Homo sapiens
atattttcagtgaaagagtt

SEQ ID NO: 8 - DNA, 3089 bp, Homo sapiens
ccttctggaaacatttgcagttacattaagtaaagtgtaaatgcacatgaatggcagcttatagagaaccaccttgtaaccagtatacaggtacaactacagctcttcagaaattggaaggttttgctagccggttatttcatagacactctaaaggtactgcacatgatcagaaaacagctctggaaaatgacagccttcatttctctgaacatactgccttatgggacagatcaatgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggttgttaaactgaagcaaatagagcatactttgaatgagaaaagaatattacaggcagtgaattttcctttccttgttcgactggagtatgcttttaaggataattctaatttatacatggttatggaatatgtccctgggggtgaaatgttttcacatctaagaagaattggaaggttcagtgagccccatgcacggttctatgcagctcagatagtgctaacattcgagtacctccattcactagacctcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaaggtccgattcccatcccacttcagttcagatctcaaggaccttctacggaacctgctgcaggtggatttgaccaagagatttggaaatctaaagaatggtgtcagtgatataaaaactcacaagtggtttgccacgacagattggattgctatttaccagaggaaggttgaagctccattcataccaaagtttagaggctctggagataccagcaactttgatgactatgaagaagaagatatccgtgtctctataacagaaaaatgtgcaaaagaatttggtgaattttaaagaggaacaagatgacatctgagctcacactcagtgtttgcactctgttgagagataaggtagagctgagaccgtccttgttgaagcagttacctagttccttcattccaacgactgagtgaggtctttattgccatcatccgtgtgcgcactctgcatccacctatgtaacaaggcaccgctaagcaagcattgtctgtgccataacacagtactagaccactttcttacttctctttgggttgtctttctcctctcctacatccatttcttccttttcaatttcattggttttctctaaacagtgctccattttattttgttggtgtttcagatgggcagtgttatggctacgtgatatttgaagggaaggataagtgttgctttcagtagttattgccaatattgttgttggtcaatggcttgaagataaactttctaataattattatttctttgagtagctcagacttggttttgccaaaactcttggtaatttttgaagatagactgtcttatcaccaaggaaatttatacaaattaagactaactttcttggaattcactattctggcaataaattttggtagactaatacagtacagctagacccagaaatttggaaggctgtagatcagaggttctagttccctttccctccttttatatcctcctctccttgagtaatgaagtgaccagcctgtgtagtgtgacaaacgtgtctcattcagcaggaaaaactaatgatatggatcatcacccagattctctcacttggtaccagcatttctgtaggtattagagaagagttctaagttttctaaaccttaactgttccttaaggattttagccagtattttaatagaacatgattaatgaaagtgacaaattttaaattttctctaatagtcctcatcataaactttttaaaggaaaataagcaaactaaaaagaacattggtttagataaatacttatactttgcaaagtcaaaaatggcttgatttttggaaacaatatagaggtattcatatttaaatgagggtttacatttgttttgttttgtaaccgttaaaaagaagttgtttccagctaattattgtggtgtactatatttgtgagcctagggtaggggcactgctgcaacttctgctttcatcccatgcctcatcaatgaggaaagggaacaaagtgtataaaacctgccacaattgtattttaattttgaggtatgatattttcagatatttcataatttctaacctctgttctctcagtaaacagaatgtctgatcgatcatgcagatacaatgttggtatttgagaggttagtttttttcctacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtcatgtaaatgctgatattgatttcactggtccatctatatttaaaacgtgc

SEQ ID NO: 9 - DNA, 2944 bp, Homo sapiens
tcacagctagcagtaagagctggtgtaattgaaagacgtttaggtgcaatcattctgctgtttgctccttgccaggttcaacatgggattgttgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggttgttaaactgaagcaaatagagcatactttgaatgagaaaagaatattacaggcagtgaattttcctttccttgttcgactggagtatgcttttaaggataattctaatttatacatggttatggaatatgtccctgggggtgaaatgttttcacatctaagaagaattggaaggttcagtgagccccatgcacggttctatgcagctcagatagtgctaacattcgagtacctccattcactagacctcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaaggtccgattcccatcccacttcagttcagatctcaaggaccttctacggaacctgctgcaggtggatttgaccaagagatttggaaatctaaagaatggtgtcagtgatataaaaactcacaagtggtttgccacgacagattggattgctatttaccagaggaaggttgaagctccattcataccaaagtttagaggctctggagataccagcaactttgatgactatgaagaagaagatatccgtgtctctataacagaaaaatgtgcaaaagaatttggtgaattttaaagaggaacaagatgacatctgagctcacactcagtgtttgcactctgttgagagataaggtagagctgagaccgtccttgttgaagcagttacctagttccttcattccaacgactgagtgaggtctttattgccatcatccgtgtgcgcactctgcatccacctatgtaacaaggcaccgctaagcaagcattgtctgtgccataacacagtactagaccactttcttacttctctttgggttgtctttctcctctcctacatccatttcttccttttcaatttcattggttttctctaaacagtgctccattttattttgttggtgtttcagatgggcagtgttatggctacgtgatatttgaagggaaggataagtgttgctttcagtagttattgccaatattgttgttggtcaatggcttgaagataaactttctaataattattatttctttgagtagctcagacttggttttgccaaaactcttggtaatttttgaagatagactgtcttatcaccaaggaaatttatacaaattaagactaactttcttggaattcactattctggcaataaattttggtagactaatacagtacagctagacccagaaatttggaaggctgtagatcagaggttctagttccctttccctccttttatatcctcctctccttgagtaatgaagtgaccagcctgtgtagtgtgacaaacgtgtctcattcagcaggaaaaactaatgatatggatcatcacccagattctctcacttggtaccagcatttctgtaggtattagagaagagttctaagttttctaaaccttaactgttccttaaggattttagccagtattttaatagaacatgattaatgaaagtgacaaattttaaattttctctaatagtcctcatcataaactttttaaaggaaaataagcaaactaaaaagaacattggtttagataaatacttatactttgcaaagtcaaaaatggcttgatttttggaaacaatatagaggtattcatatttaaatgagggtttacatttgttttgttttgtaaccgttaaaaagaagttgtttccagctaattattgtggtgtactatatttgtgagcctagggtaggggcactgctgcaacttctgctttcatcccatgcctcatcaatgaggaaagggaacaaagtgtataaaacctgccacaattgtattttaattttgaggtatgatattttcagatatttcataatttctaacctctgttctctcagtaaacagaatgtctgatcgatcatgcagatacaatgttggtatttgagaggttagtttttttcctacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtcatgtaaatgctgatattgatttcactggtccatctatatttaaaacgtgc

SEQ ID NO: 10 - DNA, 2973 bp, Homo sapiens
ctccagtgtgtgtgtttacaccatcggttcttctccctctagagattagcataactccctttgctgttggattgttattttgagcaatatgttttggaaaggttggttttcatcatgagtgtgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggttgttaaactgaagcaaatagagcatactttgaatgagaaaagaatattacaggcagtgaattttcctttccttgttcgactggagtatgcttttaaggataattctaatttatacatggttatggaatatgtccctgggggtgaaatgttttcacatctaagaagaattggaaggttcagtgagccccatgcacggttctatgcagctcagatagtgctaacattcgagtacctccattcactagacctcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaaggtccgattcccatcccacttcagttcagatctcaaggaccttctacggaacctgctgcaggtggatttgaccaagagatttggaaatctaaagaatggtgtcagtgatataaaaactcacaagtggtttgccacgacagattggattgctatttaccagaggaaggttgaagctccattcataccaaagtttagaggctctggagataccagcaactttgatgactatgaagaagaagatatccgtgtctctataacagaaaaatgtgcaaaagaatttggtgaattttaaagaggaacaagatgacatctgagctcacactcagtgtttgcactctgttgagagataaggtagagctgagaccgtccttgttgaagcagttacctagttccttcattccaacgactgagtgaggtctttattgccatcatccgtgtgcgcactctgcatccacctatgtaacaaggcaccgctaagcaagcattgtctgtgccataacacagtactagaccactttcttacttctctttgggttgtctttctcctctcctacatccatttcttccttttcaatttcattggttttctctaaacagtgctccattttattttgttggtgtttcagatgggcagtgttatggctacgtgatatttgaagggaaggataagtgttgctttcagtagttattgccaatattgttgttggtcaatggcttgaagataaactttctaataattattatttctttgagtagctcagacttggttttgccaaaactcttggtaatttttgaagatagactgtcttatcaccaaggaaatttatacaaattaagactaactttcttggaattcactattctggcaataaattttggtagactaatacagtacagctagacccagaaatttggaaggctgtagatcagaggttctagttccctttccctccttttatatcctcctctccttgagtaatgaagtgaccagcctgtgtagtgtgacaaacgtgtctcattcagcaggaaaaactaatgatatggatcatcacccagattctctcacttggtaccagcatttctgtaggtattagagaagagttctaagttttctaaaccttaactgttccttaaggattttagccagtattttaatagaacatgattaatgaaagtgacaaattttaaattttctctaatagtcctcatcataaactttttaaaggaaaataagcaaactaaaaagaacattggtttagataaatacttatactttgcaaagtcaaaaatggcttgatttttggaaacaatatagaggtattcatatttaaatgagggtttacatttgttttgttttgtaaccgttaaaaagaagttgtttccagctaattattgtggtgtactatatttgtgagcctagggtaggggcactgctgcaacttctgctttcatcccatgcctcatcaatgaggaaagggaacaaagtgtataaaacctgccacaattgtattttaattttgaggtatgatattttcagatatttcataatttctaacctctgttctctcagtaaacagaatgtctgatcgatcatgcagatacaatgttggtatttgagaggttagtttttttcctacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtcatgtaaatgctgatattgatttcactggtccatctatatttaaaacgtgc

SEQ ID NO: 11 - DNA, 3017 bp, Homo sapiens
agtgtgtgtgtttacaccatcggttcttctccctctagagattagcataactccctttgctgttggattgttattttgagcaatatgttttggaaaggttggttttcatcatgagtgcacgcaaatcatcagatgcatctgcctgctcctcttcagaaatatctgtgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggttgttaaactgaagcaaatagagcatactttgaatgagaaaagaatattacaggcagtgaattttcctttccttgttcgactggagtatgcttttaaggataattctaatttatacatggttatggaatatgtccctgggggtgaaatgttttcacatctaagaagaattggaaggttcagtgagccccatgcacggttctatgcagctcagatagtgctaacattcgagtacctccattcactagacctcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaaggtccgattcccatcccacttcagttcagatctcaaggaccttctacggaacctgctgcaggtggatttgaccaagagatttggaaatctaaagaatggtgtcagtgatataaaaactcacaagtggtttgccacgacagattggattgctatttaccagaggaaggttgaagctccattcataccaaagtttagaggctctggagataccagcaactttgatgactatgaagaagaagatatccgtgtctctataacagaaaaatgtgcaaaagaatttggtgaattttaaagaggaacaagatgacatctgagctcacactcagtgtttgcactctgttgagagataaggtagagctgagaccgtccttgttgaagcagttacctagttccttcattccaacgactgagtgaggtctttattgccatcatccgtgtgcgcactctgcatccacctatgtaacaaggcaccgctaagcaagcattgtctgtgccataacacagtactagaccactttcttacttctctttgggttgtctttctcctctcctacatccatttcttccttttcaatttcattggttttctctaaacagtgctccattttattttgttggtgtttcagatgggcagtgttatggctacgtgatatttgaagggaaggataagtgttgctttcagtagttattgccaatattgttgttggtcaatggcttgaagataaactttctaataattattatttctttgagtagctcagacttggttttgccaaaactcttggtaatttttgaagatagactgtcttatcaccaaggaaatttatacaaattaagactaactttcttggaattcactattctggcaataaattttggtagactaatacagtacagctagacccagaaatttggaaggctgtagatcagaggttctagttccctttccctccttttatatcctcctctccttgagtaatgaagtgaccagcctgtgtagtgtgacaaacgtgtctcattcagcaggaaaaactaatgatatggatcatcacccagattctctcacttggtaccagcatttctgtaggtattagagaagagttctaagttttctaaaccttaactgttccttaaggattttagccagtattttaatagaacatgattaatgaaagtgacaaattttaaattttctctaatagtcctcatcataaactttttaaaggaaaataagcaaactaaaaagaacattggtttagataaatacttatactttgcaaagtcaaaaatggcttgatttttggaaacaatatagaggtattcatatttaaatgagggtttacatttgttttgttttgtaaccgttaaaaagaagttgtttccagctaattattgtggtgtactatatttgtgagcctagggtaggggcactgctgcaacttctgctttcatcccatgcctcatcaatgaggaaagggaacaaagtgtataaaacctgccacaattgtattttaattttgaggtatgatattttcagatatttcataatttctaacctctgttctctcagtaaacagaatgtctgatcgatcatgcagatacaatgttggtatttgagaggttagtttttttcctacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtcatgtaaatgctgatattgatttcactggtccatctatatttaaaacgtgc

SEQ ID NO: 12 - DNA, 3031 bp, Homo sapiens
gctccgagtgtgtgtgtttacaccatcggttcttctccctctagagttagcataactccctttgctgttggattgttattttgagcaatatgttttggaaaggttggttttcatcatgagtgcacgcaaatcatcagatgcatctgcctgctcctcttcagaaatatctgattcctttgtgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggttgttaaactgaagcaaatagagcatactttgaatgagaaaagaatattacaggcagtgaattttcctttccttgttcgactggagtatgcttttaaggataattctaatttatacatggttatggaatatgtccctgggggtgaaatgttttcacatctaagaagaattggaaggttcagtgagccccatgcacggttctatgcagctcagatagtgctaacattcgagtacctccattcactagacctcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaaggtccgattcccatcccacttcagttcagatctcaaggaccttctacggaacctgctgcaggtggatttgaccaagagatttggaaatctaaagaatggtgtcagtgatataaaaactcacaagtggtttgccacgacagattggattgctatttaccagaggaaggttgaagctccattcataccaaagtttagaggctctggagataccagcaactttgatgactatgaagaagaagatatccgtgtctctataacagaaaaatgtgcaaaagaatttggtgaattttaaagaggaacaagatgacatctgagctcacactcagtgtttgcactctgttgagagataaggtagagctgagaccgtccttgttgaagcagttacctagttccttcattccaacgactgagtgaggtctttattgccatcatccgtgtgcgcactctgcatccacctatgtaacaaggcaccgctaagcaagcattgtctgtgccataacacagtactagaccactttcttacttctctttgggttgtctttctcctctcctacatccatttcttccttttcaatttcattggttttctctaaacagtgctccattttattttgttggtgtttcagatgggcagtgttatggctacgtgatatttgaagggaaggataagtgttgctttcagtagttattgccaatattgttgttggtcaatggcttgaagataaactttctaataattattatttctttgagtagctcagacttggttttgccaaaactcttggtaatttttgaagatagactgtcttatcaccaaggaaatttatacaaattaagactaactttcttggaattcactattctggcaataaattttggtagactaatacagtacagctagacccagaaatttggaaggctgtagatcagaggttctagttccctttccctccttttatatcctcctctccttgagtaatgaagtgaccagcctgtgtagtgtgacaaacgtgtctcattcagcaggaaaaactaatgatatggatcatcacccagattctctcacttggtaccagcatttctgtaggtattagagaagagttctaagttttctaaaccttaactgttccttaaggattttagccagtattttaatagaacatgattaatgaaagtgacaaattttaaattttctctaatagtcctcatcataaactttttaaaggaaaataagcaaactaaaaagaacattggtttagataaatacttatactttgcaaagtcaaaaatggcttgatttttggaaacaatatagaggtattcatatttaaatgagggtttacatttgttttgttttgtaaccgttaaaaagaagttgtttccagctaattattgtggtgtactatatttgtgagcctagggtaggggcactgctgcaacttctgctttcatcccatgcctcatcaatgaggaaagggaacaaagtgtataaaacctgccacaattgtattttaattttgaggtatgatattttcagatatttcataatttctaacctctgttctctcagtaaacagaatgtctgatcgatcatgcagatacaatgttggtatttgagaggttagtttttttcctacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtcatgtaaatgctgatattgatttcactggtccatctatatttaaaacgtgc

SEQ ID NO: 13 - protein, 400 aa, Homo sapiens
MAAYREPPCNQYTGTTTALQKLEGFASRLFHRHSKGTAHDQKTALENDSLHFSEHTALWDRSMKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLLKPENLLIDQQGYIQVTDFGFAKRVKGRTWTLCGTPEYLAPEIILSKGYNKAVDWWALGVLIYEMAAGYPPFFADQPIQIYEKIVSGKVRFPSSHFSSDLKDLLRNLLQVDLTKRFGNLKNGVNDIKNHKWFATTDWIAIYQRKVEAPFIPKFKGPGDTSNFDDYEEEEIRVSINEKCGKEFSEF

SEQ ID NO: 14 - protein, 357 aa, Homo sapiens
MSARKSSDASACSSSEISVMKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLLKPENLLIDQQGYIQVTDFGFAKRVKGRTWTLCGTPEYLAPEIILSKGYNKAVDWWALGVLIYEMAAGYPPFFADQPIQIYEKIVSGKVRFPSSHFSSDLKDLLRNLLQVDLTKRFGNLKNGVNDIKNHKWFATTDWIAIYQRKVEAPFIPKFKGPGDTSNFDDYEEEEIRVSINEKCGKEFSEF

SEQ ID NO: 15 - protein, 360 aa, Homo sapiens
MSARKSSDASACSSSEISDSFVMKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLLKPENLLIDQQGYIQVTDFGFAKRVKGRTWTLCGTPEYLAPEIILSKGYNKAVDWWALGVLIYEMAAGYPPFFADQPIQIYEKIVSGKVRFPSSHFSSDLKDLLRNLLQVDLTKRFGNLKNGVNDIKNHKWFATTDWIAIYQRKVEAPFIPKFKGPGDTSNFDDYEEEEIRVSINEKCGKEFSEF

SEQ ID NO: 16 - protein, 33 aa, Homo sapiens
MGNAATAKKGSEVESVKEFLAKAKEDFLKKWEN

SEQ ID NO: 17 - protein, 80 aa, Homo sapiens
MAAYREPPCNQYTGTTTALQKLEGFASRLFHRHSKGTAHDQKTALENDSLHFSEHTALWDRSMKEFLAKAKEDFLKKWEN

SEQ ID NO: 18 - protein, 21 aa, Homo sapiens
MGLLKEFLAKAKEDFLKKWEN

SEQ ID NO: 19 - protein, 20 aa, Homo sapiens
MSVKEFLAKAKEDFLKKWEN

SEQ ID NO: 20 - protein, 36 aa, Homo sapiens
MSARKSSDASACSSSEISVKEFLAKAKEDFLKKWEN

SEQ ID NO: 21 - protein, 39 aa, Homo sapiens
MSARKSSDASACSSSEISDSFVKEFLAKAKEDFLKKWEN

SEQ ID NO: 22 - protein, 4 aa, Homo sapiens
KKGS

SEQ ID NO: 23 - protein, 4 aa, Homo sapiens
RKSS

SEQ ID NO: 24 - DNA, 30 bp, Artificial Sequence
  Description of Artificial Sequence Synthetic primer
caacccaaagagaagtaagaaagtggtcta

SEQ ID NO: 25 - DNA, 30 bp, Artificial Sequence
  Description of Artificial Sequence Synthetic primer
ttggttggtctgcaaagaatgggggatagc

SEQ ID NO: 26 - DNA, 29 bp, Artificial Sequence
  Description of Artificial Sequence Synthetic primer
ttttctcattcaaagtatgctctatttgc

SEQ ID NO: 27 - DNA, 30 bp, Artificial Sequence
  Description of Artificial Sequence Synthetic primer
agaataatgccggacttgaagattttgaaa

SEQ ID NO: 28 - DNA, 27 bp, Artificial Sequence
  Description of Artificial Sequence Synthetic primer
gtttaggtgcaatcattctgctgtttg

SEQ ID NO: 29 - DNA, 27 bp, Artificial Sequence
  Description of Artificial Sequence Synthetic primer
aaaaagtcttctttggctttggctaga

SEQ ID NO: 30 - DNA, 24 bp, Artificial Sequence
  Description of Artificial Sequence Synthetic primer
tggcagcttatagagaaccacctt

SEQ ID NO: 31 - DNA, 22 bp, Artificial Sequence
  Description of Artificial Sequence Synthetic primer
caatcccatgttgaacctggca

SEQ ID NO: 32 - DNA, 24 bp, Artificial Sequence
  Description of Artificial Sequence Synthetic primer
gctctccacctcgctgcctttctt

SEQ ID NO: 33 - DNA, 24 bp, Artificial Sequence
  Description of Artificial Sequence Synthetic primer
ccagccccccttcccttccctgac

SEQ ID NO: 34 - DNA, 24 bp, Artificial Sequence
  Description of Artificial Sequence Synthetic primer
attgatctgtccataaggcagtat

SEQ ID NO: 35 - DNA, 22 bp, Artificial Sequence
  Description of Artificial Sequence Synthetic primer
tcacagctagcagtaagagctg

SEQ ID NO: 36 - DNA, 22 bp, Artificial Sequence
  Description of Artificial Sequence Synthetic primer
tctccagtgtgtgtgtttacac

SEQ ID NO: 37 - DNA, 22 bp, Artificial Sequence
  Description of Artificial Sequence Synthetic primer
atgatgaaaaccaacctttcca

SEQ ID NO: 38 - DNA, 46 bp, Artificial Sequence
  Description of Artificial Sequence Synthetic primer
gatatttctgaagaggagcaagcagatgcatctgatgatttgcgtg

SEQ ID NO: 39 - DNA, 46 bp, Artificial Sequence
  Description of Artificial Sequence Synthetic primer
cacgcaaatcatcagatgcatctgcttgctcctcttcagaaatatc

(19) United States
(12) Patent Application Publication
(10) Pub. No.: US 2004/0152626 A1
(43) Pub. Date: Aug. 5, 2004

(54) Identification of novel splice variants of the human catalytic subunit cbeta of camp-dependent protein kinase and the use thereof

(76) Inventors: Sigurd Orstavik, Oslo (NO); Nils Reinton, Oslo (NO); Eirik Frengen, Oslo (NO); Bjorn Tore Langeland, Oslo (NO); Tore Jahnsen, Oslo (NO); Bjorn S Skalhegg, Sandvika (NO)

Correspondence Address: YOUNG & THOMPSON, 745 SOUTH 23RD STREET 2ND FLOOR, ARLINGTON, VA, 22202

(21) Appl. No.: 10/168,804

(22) PCT Filed: Dec. 22, 2000
(86) PCT No.: PCT/NO00/00445

(30) Foreign Application Priority Data
Dec. 23, 1999 (NO) 19996423

(51) Int. Cl. C12Q 1/68; C07H 21/04; A61K 38/45; C12N 9/12

(52) U.S. Cl. 514/12; 435/194; 435/69.1; 435/320.1; 435/325; 435/6; 536/23.2

(57) ABSTRACT

The Cβ gene encodes at least 6 different gene products, designated Cβ1, Cβ2, Cβ3, Cβ4, Cβ4ab and Cβ4abc. As is the case with the murine and bovine splice variants, all the human Cβ splice variants vary in the N-terminal part preceding the part encoded by exon 2. Homologues to all Cβ splice variants identified in mouse and bovine were identified in human (Cβ1, Cβ2, Cβ3 and Cβ4) in addition to two novel Cβ splice variants (Cβ4ab and Cβ4abc), that have previously not been identified in any other species. Genomic DNA- and cDNA sequences encode splice variants and include the nucleotide sequences shown in SEQ ID NO: 1, 2, 3, 4, 5 and 6 respectively. The proteins are new splice variants of the Cβ protein.

### FIELD OF THE INVENTION

The present invention relates to genomic- and complementary DNA sequences encoding the 6 different gene products, designated Cβ1, Cβ2, Cβ3, Cβ4, Cβ4ab and Cβ4abc which are novel splice variants of Cβ. The present invention also relates to vectors comprising said DNA sequences and is also directed to said proteins in diagnosis and treatment.

### BACKGROUND OF THE INVENTION

Cyclic 3′,5′-adenosine monophosphate (cAMP) is a key intracellular signalling molecule, which main function is to activate the cAMP-dependent protein kinases (PKA) [1]. PKA consists of a heterotetramere, with a regulatory (R) subunit dimer and two catalytic (C) subunits. The holoenzyme is activated when four molecules of cAMP bind to the R subunit dimer, two to each R subunit, releasing two free active C subunits [2]. In man, four different R subunits (RIα, RIβ RIIαt, RIIβ), and four different C subunits (Cα, Cβ, Cγ and PrKX) have been identified [3]. The Cα and Cβ subunits are expressed in most tissues, while the Cγ subunit, which is transcribed from an intron-less gene and represents a retroposon derived from the Cα subunit [4], is only expressed in human testis [5]. PrKX is an X chromosome-encoded protein kinase, and was recently identified as a PKA C subunit since it is inhibited by both PKI and RIα and the RIα/PrKX complex is activated by cAMP [6].

Splice variants of both Cα and Cβ have been identified. The splice variants of Cα have been termed Cα1 (previously named Cα [7]), Cα2 [8] and Cα-s [9]. Originally Cα2 was isolated from interferon-treated cells and identified as a C-terminally truncated Cα1 subunit. However, recently a novel Cα2 splice variant was reported [10]. The novel Cα2 variant was shown to be identical to the previously identified Cα splice variant, Cα-s. Moreover, Cα-s which was originally isolated and characterized from ovine sperm [9], has later been cloned from a human testis cDNA library and identified in human sperm [11]. Both Cα-s/Cα2 are encoded with a truncated N-terminal end when compared to Cα1. The variable parts of Cα1 and Cα-s are located upstream of exon 2 in the murine Cα gene, implying that the variation in the N-terminal end of the Cα1 and Cα-s/Cα2 are due to alternative use of different first exons. In bovine, two splice variants of Cβ have been identified, termed bovine Cβ1 [12] and bovine Cβ2 [13]. The bovine splice variants contain variable N-terminal ends in which the non-identical sequences are most probably encoded by different forms of exon 1. Bovine Cβ2 is expressed at low levels in most tissues with the highest expression in the spleen, thymus, and kidney and to some extent brain. Furthermore, in the mouse, three splice variants of Cβ have been identified and are designated mouse Cβ1, mCβ2 and mouse Cβ3 [14]. Whereas mouse Cβ1 is ubiquitously expressed, mouse Cβ2 and mouse Cβ3 have so far only been identified in the brain. The mouse Cβ1 and bovine Cβ1 are similar in the entire sequence, demonstrating that they represent orthologe protein sequences. However, neither mouse Cβ3 nor mouse Cβ4 were similar to bovine Cβ2 in the N-terminal part, indicating that their N-terminals are encoded by unrelated exons. Previous to this study, only a single splice variant of human Cβ had been identified (Cβ1), homologous to mouse Cβ1 and bovine Cβ1.

### SUMMARY OF THE INVENTION

The present invnetion demonstrate that the Cβ gene encodes at least 6 different gene products, designated Cβ1, Cβ2, Cβ3, Cβ4, Cβ4ab and Cβ4abc. As is the case with the murine and bovine splice variants, all the human Cβ splice variants vary in the N-terminal part preceding the part encoded by exon 2. Homologues to all Cβ splice variants identified in mouse and bovine were identified in human (Cβ1, Cβ2, Cβ3 and Cβ4) in addition to two novel Cβ splice variants (Cβ4ab and Cβ4abc), that have previously not been identified in any other species. The present invention includes in this respect genomic DNA- and cDNA sequences encoding said splice variants and comprises the nucleotide sequences shown in SEQ ID NO: 1, 2, 3, 4, 5 and 6 respectively. Wherein the said proteins are new splice variants of the Cβ protein. The present invention is further directed to vectors comprising said cDNA sequences. The invention also includes proteins characterised by the specific amino acid Cβ splice variant proteins Cβ2, Cβ4ab and Cβ4abc shown in SEQ ID NO: 7, 8 and 9. The invention includes further use of the said Cβ splice variant proteins and DNA sequences in preparation of pharmaceuticals for diagnostic- and therapeutic purposes.

### BRIEF DESCRIPTION OF THE DRAWINGS

FIG. 1: A: Identification of cDNAs encoding human Cβ splice variants. Schematic representation of the protein-encoding sequences of the various Cβ splice variants found in human. Human cDNAs from total fetus and brain were amplified using primers complementary to the Cβ cDNA, subcloned and sequenced. The resulting cDNAs were identical to the previously published Cβ cDNA (Cβ1) downstream of nucleotide 46 (constant region). However, five novel cDNA sequences, designated Cβ2, Cβ3, Cβ4, Cβ4ab and Cβ4abc, could be identified based on differences in the 5′-ends of the sequences (variable region).

FIG. 2: A: Structure of the human genomic region encoding the novel Cβ splice variants. Primers were made based on exon 2 and the most 5′-end of the different Cβ cDNAs, and used to amplify human genomic DNA by PCR. Two overlapping PCR products of 14 and 17 kb, respectively, were identified and mapped by Southern blotting and hybridization to oligonucleotides corresponding to the different cDNAs. As derived from the 14 and 17 kb PCR products, exon 1-2 1-3,1-4 and exon a, b and c are located 31, 14.1, 14, 8.1, 5.4 and 4.4 kb upstream of exon 2. Based on restriction mapping of the PAC clone RPCI-6-228E23, exon 1-1 is located approximately 60 kb upstream of exon 1-2. Exon 1-1 is specific for the splice variant, which encodes Cβ1. The exons are indicated as vertical lines. The introns are drawn to scale as indicated. B: Nucleotide sequence of genomic regions encoding novel splice variants of Cβ. Protein encoding sequences are in capital letters, intron and 5′-untranslated sequences are in lower case letters. Translation initiation codons are underlined. Only the 5′-end of exon 2 is included. C: Schematic representation of how the various human Cβ exons 5′ to exon 2 may be spliced. The upper panel describes a potential model in which four variants of exon 1 designated exon 1-1,1-2, 1-3 and 14 may alternatively splice with exon 1 to encode the splice variant specific sequence in Cβ1, Cβ2, Cβ3 and Cβ4. The lower panel describes a model in which the exons a, b and c may splice with exon 14 and 1-3 upstream of exon 2 to encode the splice variant-specific sequences in Cβ4ab, Cβ4abc and Cβ3ab.

FIG. 3: Deduced amino acid sequence of Cβ splice variants. The amino acid sequences of the amino terminal parts of Cβ1 and five new splice variants, designated Cβ2, Cβ3, Cβ4, Cβ4ab and C4βabc according to the cDNA clones shown in FIG. 1A. The amino acid sequences are shown in the one letter code and demonstrate that six novel Cβ exons give rise to five different cDNAs as a result of alternative promoter use and alternative splicing. The myristylation motive G-N previously identified in Cβ1 is boxed. A PKA autophosphorylation motive that has previously been identified in Cβ1, is underlined and Ser10 which is potentially phosphorylated, is labeled by an asterisk. Note that there is a PKA autophosphorylation motif, encoded by exon a, present in Cβ4ab and Cβ4abc.

FIG. 4: Tissue distribution of different Cβ splice variants. Northern blots containing various human tissues were hybridized using probes specific for Cβ1, Cβ2, Cβ4, exon a+b and a probe common to all Cβ splice variants (Cβ common). For comparison, the same blots were hybridized using a GAPDH cDNA (GAPDH). All Cβ1 mRNAs had the same apparent length (4.4 kb).

FIG. 5: A: Species distribution of Cβ2. A Southern blot containing EcoRI digested genomic DNA from various species was hybridized using a DNA probe corresponding to exon 1-2 (Cβ2 specific). A single hybridizing band identifying genomic sequence homologous to human exon 1-2 was identified in mammalians such as monkey, dog, rabbit and human except mouse and rat. B: Cβ2 is not expressed in the mouse. A Northern blot containing total RNA (20 μg pr. lane) isolated from wild type (+/+) mouse brain and spleen (lane 1 and 3), brain and spleen of mice ablated (−/−) for Cβ1 (lane 2 and 4) and human peripheral blood leukocytes (lane 5) was probed with a Cβ probe expected to recognize all known Cβ splice variants (Co Common, upper panel) and a Cβ probe specific for the Cβ2 splice variant (Cβ2, lower panel). Messenger RNA recognized by the two probes is indicated as 4.4 kb.

### DETAILED DESCRIPTION OF THE INVENTION

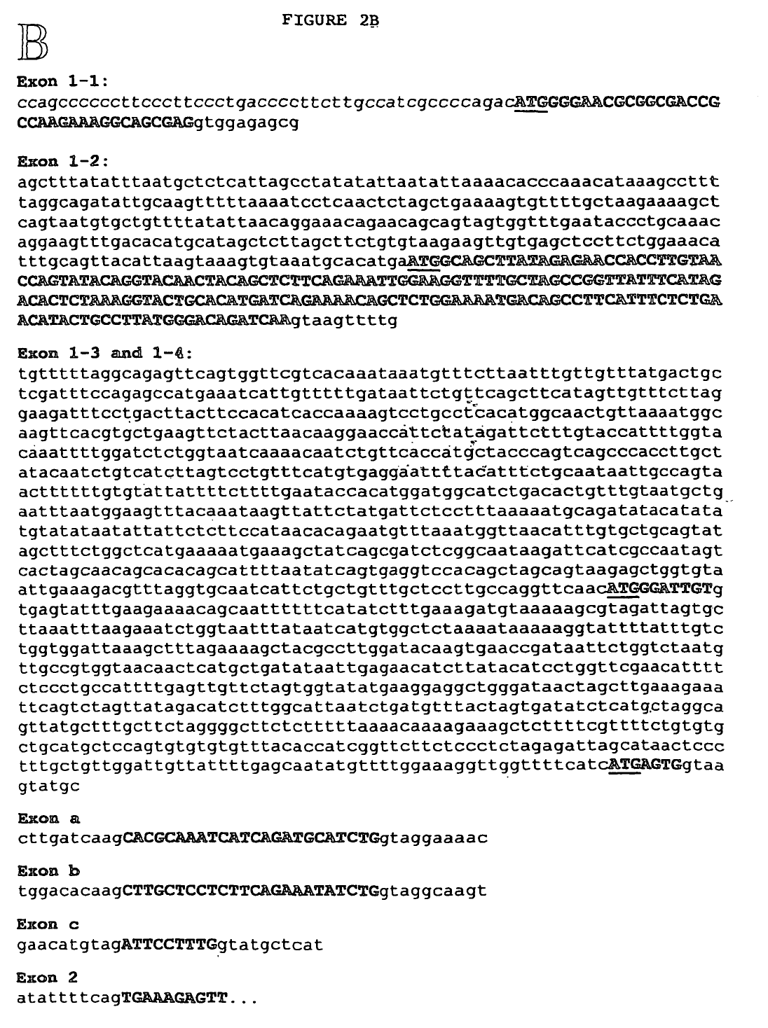

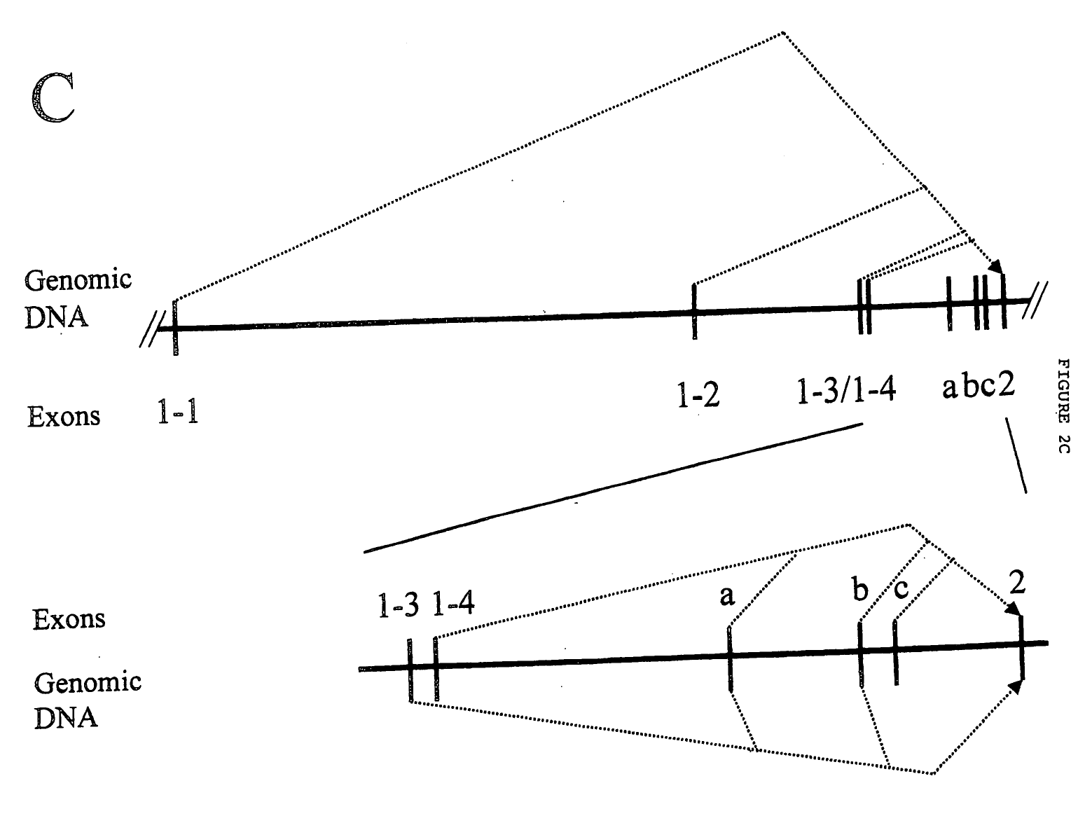

The present invnetion demonstrate that the human Cβ gene encodes five novel Cβ splice variants, designated Cβ2, Cβ3, Cβ4, Cβ4ab and Cβ4abc, in addition to the previously identified splice variant Cβ1 [12]. All the Cβ splice variants contained a unique N-terminal end, and showed tissue specific expression. As we found no evidence of an additional exon upstream of exon 1-1 and all the cDNA characterized had unique 5′-ends, it is reasonable to assume that the exon 1-1,1-2, 1-3 and 14 each contain a separate promoter, and that the resulting mRNA products are due to alternative use of different promoters. Despite this, we can not rule out the possibility that two or more of these splice variants share a common promoter used to alternatively splice the different exons. Furthermore, we found two Cβ variants, Cβ4ab and Cβ4abc, that were the results of alternative splicing of either exon a and b, or exon a, b and c, between exon 1-4 and exon 2. The presence of the corresponding mRNA was confirmed by hybridizing a Northern blot with a probe complimentary to the sequences found in exons a and b. This probe and the probe specific for Cβ4 bound to an RNA with the same apparent length located in human brain. The location of the exons a, b and c may suggest that they generate splice variants of Cβ in addition to those demonstrated here. Indeed, a short cDNA from human infant brain have been sequenced and demonstrated to contain a combination of exons 1-3, a, b and 2 (Accession no. AA35 1487, see FIG. 2C). We were unable to produce such a cDNA, which could be due to low level expression of Cβ3 in adult brain.

The two splice variants Cα1 and Cβ1 are highly conserved in the parts encoded by exon 1, differing in only 2 of the first 16 amino acids [7;12]. It is therefore tempting to suggest that this region serve a specific role in the function of these splice variants. Thus, the fact that we have identified several Cβ splice variants with variable N-terminal ends could suggest that the N-terminal domain might reflect specific functional features associated with each splice variant. This is supported by studies of the mouse Cβ1 KO mouse, which displayed impaired hippocampal plasticity [16]. However, to what extent N-terminal differences influence catalytic activity is not known since it was shown that the N-terminally truncated Cβ splice variants in mouse, Cβ2 and Cβ3 were catalytically active, an activity that was inhibited both by PKI and the R subunit in vivo [14]. In addition, a study by Herberg et al [17] showed that deleting amino acids 1-14 in the Cβ isoform did not influence catalytic activity, demonstrating that the N-terminal specific for the Cα1/Cβ1 is not necessary for catalytic activity.

The N-terminal of Cα1 and Cβ1 contain two sites for post-translational modification, a myristylation site and an autophosphorylation site [5;18;19]. In Cα1, Cβ1 and Cβ3 the N-terminal amino acid is G (Gly) which has been shown as an absolute requirement for myristylation [20]. Despite this, it was previously demonstrated in the mouse that Cβ3 does not undergo myristylation in vivo [14]. This phenomena may be explained based on a recent study, demonstrating that the amino acid C-terminal to G must be N if myristylation shall occur. This because deamination of N to yield D is an absolute requirement [21]. Because the amino acid C-terminal to G is L in both mouse and human Cβ3, it explains why mouse Cβ3 is not myristylated and suggests that the human Cβ3 may not be myristylated in vivo.

The fact that several human Cβ splice variants (Cβ2, Cβ3, Cβ4, Cβ4ab and Cβ4abc) lack the ability to become myristylated in vivo, question the role of this post translational modification. Based on the Ca crystal structure it appears that the myristyl group serves to fill and shade a hydrophobic pocket in the large lobe [22], suggesting that this N-terminal modification serves to solubilize the C subunit. This is supported by two independent observations. Firstly, expression of an N-terminally truncated form of Cα1 revealed a C subunit tightly associated with the particulate fraction [23]. Secondly, the Cα-s/Cα2 which is a naturally occuring N-terminally truncated splice variant is tightly associate with sub cellular structures in both ovine-[9,24] and human [11] sperm. This taken together with a recent report, which demonstrated that the myristyl group serves to increase the lipofilic properties of the C subunit when binding the RII- but not the RI subunit [25], suggests that the N-terminal amino acids of Cα1 together with myristylation serves to influence C subunit solubility. Thus, the sequence similarity between Cα1 and Cβ1 and the difference in solubility of Cα1 and Cα-s/Cα2, may imply comparable difference in solubility between Cβ1 and the truncated Cβ forms.

Previously a consensus autophosphorylation motif (-KKGS¹⁰-) was identified in Cα1 and Cα1 [12;26], that is phosphorylated when Cα1 is expressed in bacteria [18;23]. In the study by Yonemoto et al. (1993) mutation of S¹⁰ yielded an insoluble enzyme that appeared inactive. Thus, the N-terminal domain may also have implications for catalytic activity by an unknown mechanism. However, like the human Cβ2, Cβ3, Cβ4, the mouse Cβ2 and Cβ3 lack S¹⁰, yet these splice variants are soluble and catalytically active in vivo [14]. This suggests that the human homologues most probably are active and may imply that S¹⁰ phosphorylation is not crucial for C subunit catalysis. Interestingly, we identified a potential autophosphorylation site (-RKSS⁶-) in Cβ4ab and Cβ4abc that was encoded by exon a. To what extent this site represents a true autophosphorylation site that will influence Cβ4ab and Cβ4abc properties, remains to be seen.

The human Cβ2 splice variant was similar to the previously identified bovine Cβ2 splice variant, but we have been unable to identify a similar splice variant in mice. Interestingly, the human Cβ2 splice variant is expressed only in peripheral tissues, while no detectable Cβ2 mRNA signal is found in human brain. However, no Cβ can be detected outside the brain in mice lacking the Cβ1 splice variant [14;16]. In addition, we were unable to detect any signal when hybridizing mouse DNA using a human Cβ2 specific probe. Thus, it is likely that mice do not contain a homologue of the human and bovine Cβ2 splice variants.

Interestingly, Cβ2 is the most a typical of the Cβ splice variants. This subunit is encoded with an extended N-terminal domain, which do not resemble any of the other Cβ splice variants. The unique domain together with the fact that Cβ2 lacks the myristylation- as well the autophosphorylation site, and that Cβ2 is the only Cβ splice variant not identified in the brain, may suggest specific and unique features associated with this splice variant in other tissues that will await further studies.

The inventors suggest that tissue-specific expression of various Cβ splice variants when complexed with R subunits may imply novel PKA holoenzymes with specific functional features that may be important as mediators of cAMP effects.

The present invention includes in this respect genomic DNA- and cDNA sequences encoding splice variants Cβ1, Cβ2, Cβ3, Cβ4, Cβ4ab and Cβ4abc and comprises the nucleotide sequence shown in SEQ ID NO: 1, 2, 3, 4, 5 and 6 respectively. Wherein the said proteins are new splice variants of the Cβ protein. The present invention is further directed to vectors comprising said cDNA sequences. The invention also includes proteins characterised by the specific amino acid Cβ splice variant proteins; Cβ2, Cβ4ab and Cβ4abc shown in SEQ ID NO: 7, 8 and 9 respectively. The invention includes further use of the said Cβ splice variant proteins and DNA sequences in preparation of pharmaceuticals for diagnostic- and therapeutic in order to identify, characterize and produce pharmacological compositions.

Cβ2 is an enzyme that is expressed in lymphoid cells, whereby its function is to mediate the regulatory effects of cAMP on T cell activation. Thus, altered levels, location and/or activity of Cβ2 will according to the inventors results, have impact on the regulation and normal function of receptors and enzymes which are important for T cell activation and are regulated by cAMP. This knowledge can be used to diagnose hyperreactive and dysfunctional T cells associated with various immune diseases.

1) Malfunctioned T cells: I is well known that T cells isolated from patients suffering from T cell-dependent common variable immune deficiency (CVI) and acquired immune deficiency syndrome (AIDS) do not respond to antigen. Furthermore, T cells isolated from patients suffering from certain types of rheumatoid arthritis and other auto immune diseases are hyper sensitive to foreign antigens. In both cases these situations evoke abnormal immune responses that may involve malfunctioned Cβ2. This may either be monitored as constitutively activated Cβ2, sub-normal activity or dislocation of Cβ2.

1.1) Improving T cell dysfunction: Present invention makes it possible to identify, characterize and produce pharmacological compositions after high through put screening that specifically will inhibit the enzymatic activity of Cβ2. These compositions should be developed such that they can be introduced orally or intra venously to enter the blood system reaching the dysfunctional T cells.

Furthermore, dislocation of Cβ2 protein from the T cell membrane will short cut the regulatory effects of Cβ2 on relevant receptors. Thus, the present invention makes it possible to identify, characterize and produce pharmacological composition after high through put screening that will specifically and irreversibly block Cβ2 interaction with the T cell membrane. These compositions should be developed such that they can be introduced orally or intra venously to enter the blood system reaching the T cell.

1.2) Down regulation of hyper active T cells: Present invention makes it possible to identify, characterize and produce pharmacological compositions after high through put screening that specifically will activate the enzymatic activity of Cβ2. These compositions should be developed such that they can be introduced orally or intra venously to enter the blood system reaching the dysfunctional T cells.

1.3) Kits for diagnosing Cβ62 mutations: T cell malfunction caused by mal function or -localization of Cβ2 enzyme activity may be caused by mutation(s) in the Cβ2 protein. Present invention makes it possible to develop kits, which would diagnostically facilitate if mutated Cβ2 is present. Such kits should be developed with Cβ2 specific DNA probes.

Present invention makes it possible to develop a method for inspection and screening of patient T cells for the presence and location of Cβ2 comprising:

a) collection and washing in buffer of isolated peripheral blood T lymphocytes according to [27];

b) preparing for identification of Cβ2 protein by immunofluorescence, T cells are let to settle onto poly L-lysine coated cover slips following detergent-dependent lysis;

c) incubation with primary antibody (Ab), either irrelevant Ab or Cβ2 specific Ab, Ab overshoot will be removed by washing buffer and T cells incubated with secondary anti-IgG Ab conjugated with a fluorescent;

d) inspection of T cells under fluorescent microscopy.

Present invention makes it further possible to develop a method of screening patient T cells for membrane associated Cβ2 catalytic activity comprising:

a) collection and washing in buffer of isolated peripheral blood T lymphocytes according to [27];

b) preparation of T cells by lysing in detergent buffer;

b) monitoring Cβ2 specific catalytic activity by established assay, Cβ1 activity is used as an internal control to determine relative activity.

Present invention makes it also possible to screen patients for mutations in the Cβ2 gene and mRNA comprising:

a) collection and washing in buffer of isolated peripheral blood T lymphocytes according to [27];

b) isolation of total RNA and genomic DNA according to established methods followed by RT-PCR using Cβ2 specific primers according to cDNA sequence of Cβ2 specific nucleotides or the Cβ2 specific exon, designated exon 1-2.

Materials and Methods.

General Protocols

Complementary DNA probes were radiolabeled using the Megaprime random priming kit and α-[32P]dCTP (Amersham) as instructed by the manufacturers to a specific activity of at least 1×10⁹ cpm. Synthetic oligonucleotides were radiolabeled using T4 polynucleotide kinase (Pharmacia) and γ-[32P]ATP as instructed by the manufacturer.

DNA was either sequenced manually using Thermo Sequenase radioabeled terminator cycle sequencing kit (Amersham, Buckinghamshire, UK) or by Medigenomix (Martinsried, Germany). Sequences were analyzed using the Wisconsin University GCG program package (UWGCG) and the basic local alignment and search tool (BLAST) [5].

Identification of cDNAs

The 5′-end of human Cβ cDNA was amplified from human total fetus and brain Marathon RACE-ready cDNAs (Clontech) using the Advantage KlenTaq Polymerase Mix (Clontech) as described by the manufacturer. Amplification was performed using adapter primer 1 (Clontech) and four different primers complementary to the human Cβ cDNA sequence (5′-CAACCCAAAGAGAAGTAAGAAAGTGGTCTA-3′, 5′-TTGGTTGGTCTGCAAAGAATGGGGGATAGC-3′, 5′-TTTTCTCATTCAAAGTATGCTCTATTTGC-3′ and 5′-AGAATAATGCCGGACTTGAAGATTTTGAAA-3′).

Five cycles were performed with 45 sec 94° C., 2 min 72° C., five cycles 45 sec 94° C., 2 min 70° C., 25 cycles 45 sec 94° C., 2 min 68° C., and a final extension of 10 min at 72° C. The resulting products were separated by gel electrophoresis, subcloned to pCR2.1TOPO (Invitrogen) as instructed by the manufacturer and sequenced.

Amplification of Cβ gene fragments.

A genomic fragment was amplified using an oligonucleotide corresponding to exon 1-3 (5′-GTTTAGGTGCAATCATTCTGCTGTTTG-3′) and a primer complementary to sequences in exon 2 (5′-AAAAAGTCTTCTTTGGCTTTGGCTAGA-3′). Another genomic fragment was amplified using a primer corresponding to exon 1-2 (5′-TGGCAGCTTATAGAGAACCACCTT-3′) and a primer complementary to sequence found in exon 1-3 (5′-CAATCCCATGTTGAACCTGGCA-3′). PCR reactions were performed using the Boehringer-Mannheim Expand Long Template PCR kit as instructed by the manufacturer using buffer 2. PCR was performed using human genomic DNA (Boehringer-Mannheim) as template with 1 min at 92° C., 30 cycles of 10 sec 94° C., 30 sec 60° C. and 10 min (extended with 20 sec per cycle from cycle 11 to cycle 30) 68° C., and a final incubation of 7 min at 68° C. Products were separated by agarose gel electrophoresis and analyzed by Southern blotting using radiolabeled cDNAs and synthetic oligonucleotides corresponding to the different exons.

Screening of PAC Library and Subcloning of Exon-containing Sequences.

The human P1-derived Artificial Chromosome (PAC) library, RPCI-6 was screened and the isolated bacterial clone was grown in liquid culture and plasmid DNA was isolated using ion-exchange columns as described by the manufacturer (Qiagen, Hilden, Germany). Exon-containing DNA restriction fragments were identified by Southern blotting using radio labeled cDNAs and synthetic oligonucleotides. Exon-containing fragments were excised from the gel and subcloned to the pZERO2.1 vector (Invitrogen) as instructed by the manufacturer.

Generation of Splice Variant Specific Probes, Northern Blotting and Southern Blotting.

DNA fragments corresponding to the splice variant-specific parts of the cDNAs were amplified by PCR. The following primers were used for the different splice variants:

| | |
|---|---|
| Cβ1: | 5′-GCTCTCCACCTCGCTGCCTTTCTT-3′ |
| | and |
| | primer 5′-CCAGCCCCCCTTCCCTTCCCTGAC-3′, |
| Cβ2: | primer 5′-TGGCAGCTTATAGAGAACCACCTT-3′ |
| | and |
| | primer 5′-ATTGATCTGTCCATAAGGCAGTAT-3′, |
| Cβ3: | primer 5′-TCACAGCTAGCAGTAAGAGCTG-3′ |
| | and |
| | primer 5′-CAATCCCATGTTGAACCTGGCA-3′, |
| Cβ4: | primer 5′-TCTCCAGTGTGTGTGTTTACAC-3′ |
| | and |
| | primer 5′-ATGATGAAAACCAACCTTTCCA-3′. |

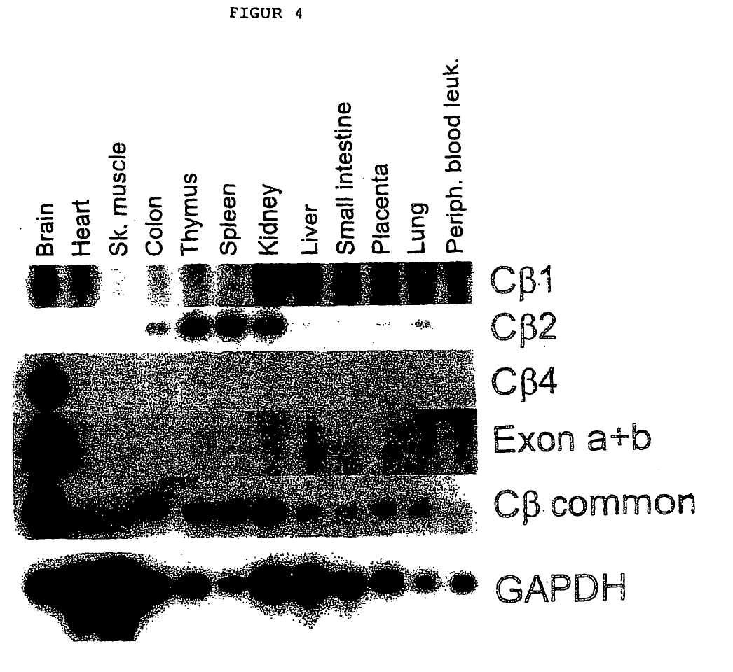

The primers were used for amplification of the fragments from cloned RACE-products using Taq DNA polymerase (Perkin-Elmer) as described by the manufacturer. For generation of a probe specifically recognizing exon a and b, the primers 5′-GATATTTCTGAAGAGGAGCAAGCAGATGCATCTGATGATTTGCGTG-3′ and 5′-CACGCAAATCATCAGATGCATCTGCTTGCTCCTCTTCAGAAATATC-3′ were annealed, phosphorylated and ligated. A 1.5 kb fragment of Cβ cDNA [5] was used for recognizing the parts of the Cβ mRNA common to all splice variants. Two similar Northern blots containing RNA from various human sources were purchased from Clontech. One blot was hybridized using a probe specific for Cβ2, while the other blot was probed in succession with probes specific for Cβ3, Cβ4, exon a and b, and the 1.5 kb Cβ cDNA. Both blots were hybridized using GAPDH cDNA as control. As an almost identical pattern of hybridization was obtained using GAPDH on both blots, only one GAPDH blot is shown (FIG. 4). All probes were hybridized in ExpressHyb hybridization solution (Clontech) as described by the manufacturer. A Southern blot containing EcoRI-digested DNA from various species (Clontech) and Southern blots containing human and mouse DNA digested with various enzymes were hybridized using the probe specific for Cβ2. The filters were prehybridized in 5× Denhardt's solution, 5×SSC, 50 mM sodium phosphate buffer, pH 6.8, 0.1% SDS, 250 μg/ml single stranded salmon sperm DNA, and 50% (v/v) formamide at 42° C. for 3 h, and hybridized for 16 h in a similar solution containing the radiolabeled Cβ common or Cβ2 probe. The membranes were washed four times in 2×SSC, 0.1% SDS for 5 min at room temperature, followed by two washes using 0.5×SSC, 0.1% SDS at 50° C. for 30 min. Autoradiography was performed at −70° C. using Amersham Hyperfilm MP and intensifying screens.

In order that this invention may be better understood, the following examples are set forth. These examples are for the purposes of illustration only, and are not to be construed as limiting the scope of the invention in any manner.

### EXAMPLES

### Example 1

Identification of Exons Encoding Novel Splice Variants of Human Cβ.

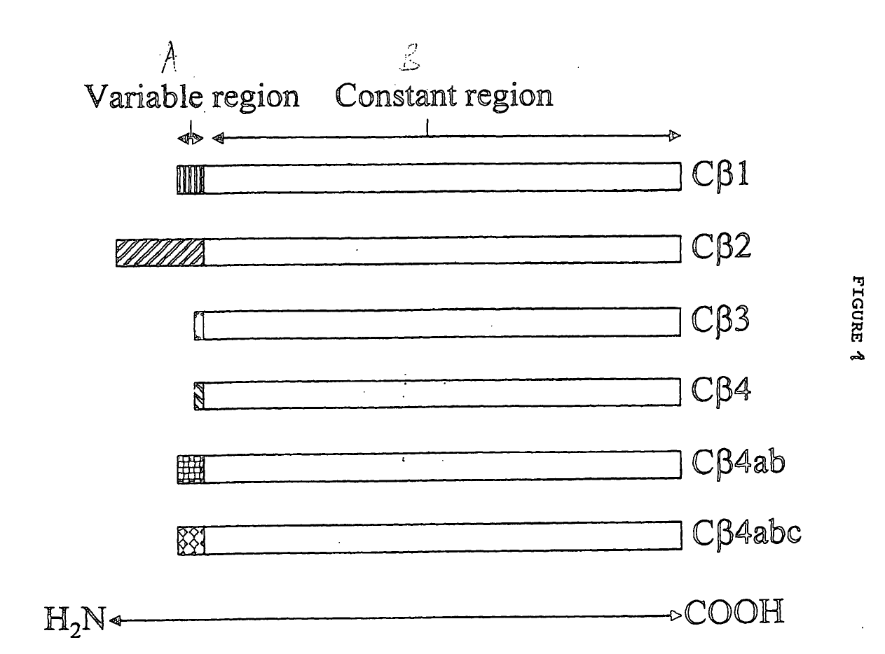

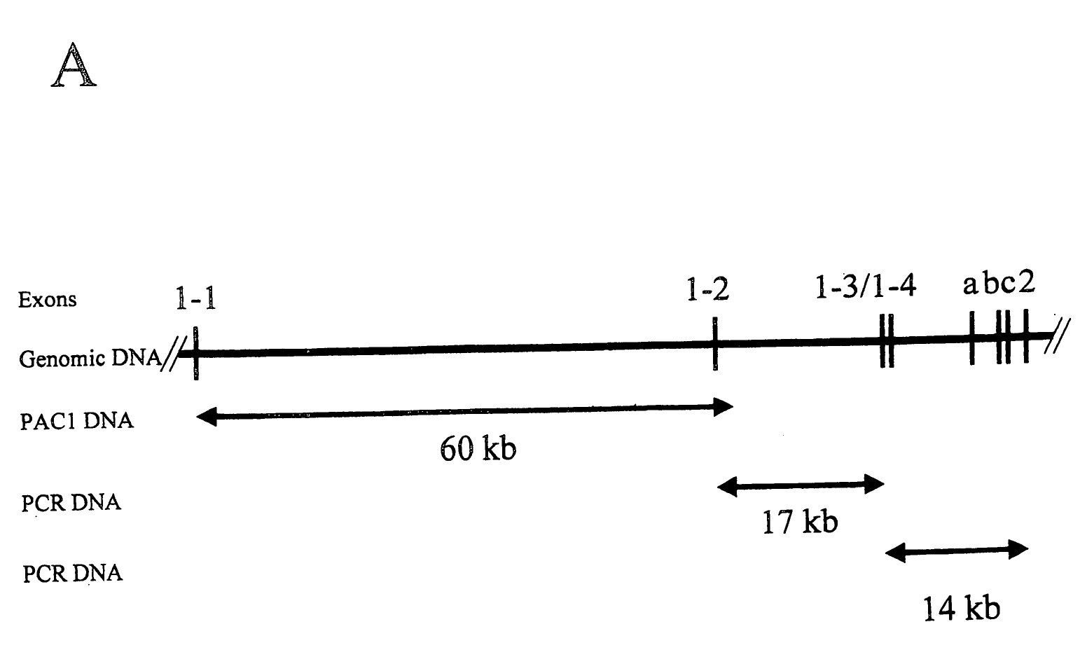

The 5′-ends of human Cβ cDNAs were amplified from human brain and total fetus RACE-ready cDNA using four different oligonucleotide primers complementary to the previously published human Cβ cDNA sequence, in combination with an anchor primer. The resulting PCR products were subcloned, sequenced and compared to the previously published human CβαcDNA sequence which is now designated Cβ1 (FIG. 1). All clones sequenced were shown to lack the 46 first protein-encoding nucleotides in the human Cβ1 cDNA sequence. Instead 5 novel stretches of protein encoding sequences were identified (FIG. 1, variable region). Each of the clones contained a translation initiation codon and one or more in-frame upstream stop codons. The five novel cDNA sequences were designated Cβ2, Cβ3, Cβ4, Cβ4ab and Cβ4abc.

All the Cβ cDNAs were similar from nucleotide 47 and down stream in the Cβ1 cDNA, which corresponds to the start of exon 2 in the murine Cβ gene. The identification of novel protein-encoding sequences upstream of exon 2, indicated the presence of several different exons upstream of exon 2. Thus, human genomic DNA was amplified using a combination of primers corresponding to exon 2 (antisense orientation) and the 5′-ends of the different novel cDNAs (sense and antisense orientation) in different combinations. A 17 kb PCR product was the result of an amplification using a primer corresponding to the 5′-end of Cβ2 cDNA (sense orientation) and the 5′-end of Cβ3 (antisense orientation) Furthermore, a 14 kb PCR product was the result of an amplification using a primer corresponding to the 5′-end of Cβ3 cDNA (sense orientation) and a primer corresponding to exon 2 (antisense orientation). These clones enabled us to physically map six novel exons in the Cβ gene that were designated 1-2,1-3, 1-4, a, b and c, and which were located 31, 14.1, 14, 8.1, 5.4 and 4.4 kb upstream of exon 2, respectively (FIG. 2A). Furthermore, a PAC library was screened using the 5′ ends of Cβ1 and Cβ2 cDNAs as probes. One of the clones identified, RPCI-6-228E23, contained both exon 1-2 and an exon containing the entire splice variant-specific part of the Cβ1 cDNA, which we termed exon 1-1. This PAC clone was selected for detailed restriction mapping using CpG cutters. The digested PAC DNA was separated by pulsed-field gel electrophoresis (PFGE), transferred to Southern blot membranes and hybridized with exon 1-1 and 1-2, as well as Sp6 and T7 oligonucleotide probes. These results revealed a distance of approximately 60 kb between exon 1-1 and 1-2 (FIG. 2A). All nucleotide sequences found in the different Cβ cDNAs could be identified in a continuous stretch of human genomic DNA, thereby supporting the notion that these cDNAs are products of the same gene. Exon 1-1 was shown to be homologous to the previously identified exon 1A of the murine Cβ gene. As shown in FIG. 2B, exon 1-2 contains the entire Cβ2 specific sequence, and exon 1-3 contains the sequence specific for Cβ3 which is homologous to the previously identified exon 1B in the mouse Cβ gene. Finally, exon 1-4 was shown to contain the sequence specific for the human Cβ4 splice variant, and to be homologous to the murine exon 1C, which encodes the N-terminal end in the murine Cβ2 splice variant. Based on the Cβ4ab and Cβ4abc cDNA sequences, the exons a, b and c (FIG. 2B), were demonstrated to be alternatively spliced in between exon 1-4 and exon 2, with either exons 1-4, a, b and 2 or exons 1-4, a, b, c and 2 (FIG. 2C, lower panel). These cDNA sequences represent novel Cβ splice variants not identified in any other species.

### Example 2

Deduced Amino Acid Sequence of Novel Cβ3 Splice Variants.

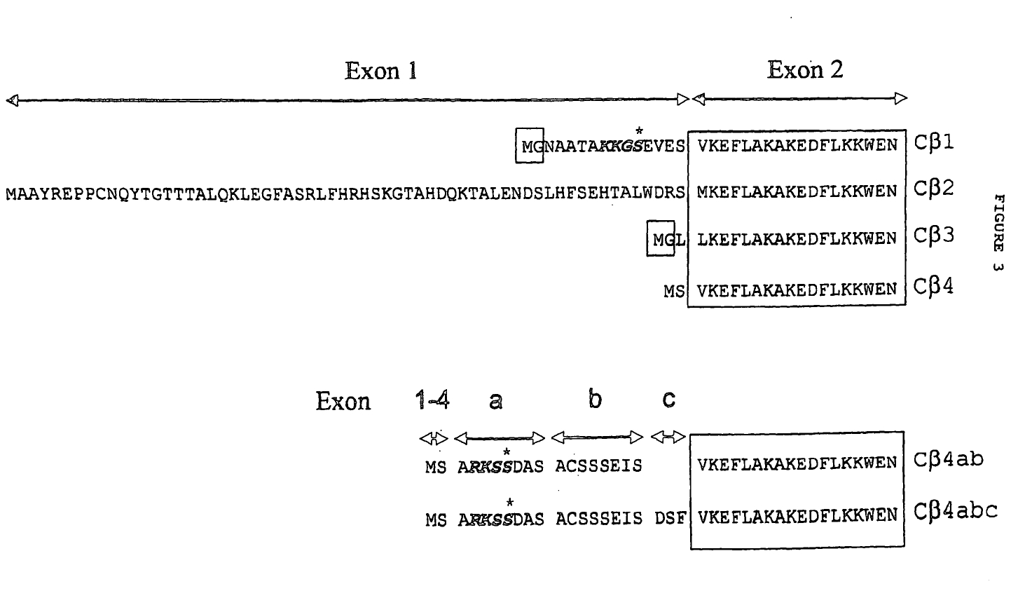

The N-terminal parts of the deduced amino acid sequences of the previously published Cβ1-sequence and the 5 novel Cβ splice variants are illustrated in FIG. 3 (upper and lower panels). The splice variants were identical starting from the sequence encoded by exon 2 (amino acid 17 in Cβ1) to the C-terminus, while the N-termini varied both in length and sequence composition. The Cβ2 splice variant contains a 63 amino acid sequence substituting the first 16 amino acids in Cβ1, and is homologous to the previously identified bovine Cβ2 [13]. Furthermore, the human Cβ3 splice variant contains four amino acids in the N-terminal substituting the first 16 amino acids in Cβ1, and is similar to the previously identified murine Cβ3 [14]. The human Cβ4 contains three amino acids substituting the first 16 amino acids in Cβ1, and is similar to the murine Cβ2 [14]. Finally, the splice variants Cβ4ab and Cβ4abc contain 18 and 21 amino acids, respectively, that substitute the first 16 amino acids of Cβ1. These splice variants show no homology to the N-terminus of any other C subunits identified thus far.

### Example 3

Tissue Distribution of Cβ Splice Variants.

To examine the tissue distribution of Cβ splice variants, exon specific DNA probes and a DNA probe common to all Cβ splice variants were hybridized to two similar Northern blots containing RNA from various human tissues. For comparison the blots were hybridized to a cDNA encoding glycer-aldehyde 3-phosphate dehydrogenase (GAPDH). In FIG. 4 (panel Cβ1) we show that Cβ1 is predominantly expressed in brain and kidney with low level expression in several other tissues as well. Cβ2 is expressed at high levels in thymus, spleen and kidney in addition to a weak signal in other tissues (FIG. 4, panel Cβ2). In contrast to Cβ2 the exon 1-4 and exon a and b containing mRNAs appeared to be present exclusively in brain (FIG. 4, panels Cβ4 and exon a+b). Finally, probing the Northern blot with a probe common to all the Cβ splice variants, we observed ubiquitous expression of Cβ with the strongest signal in brain and a somewhat weaker signal in spleen and thymus, when compared to the GAPDH signal (FIG. 4, panel Cβ common). Hybridization using a DNA fragment corresponding to the Cβ3 specific cDNA resulted in an almost undetectable signal in the brain and no detectable signals in any other tissues (data not shown).

### Example 4

The Human Cβ2 Splice Variant is Not Present in the Mouse.

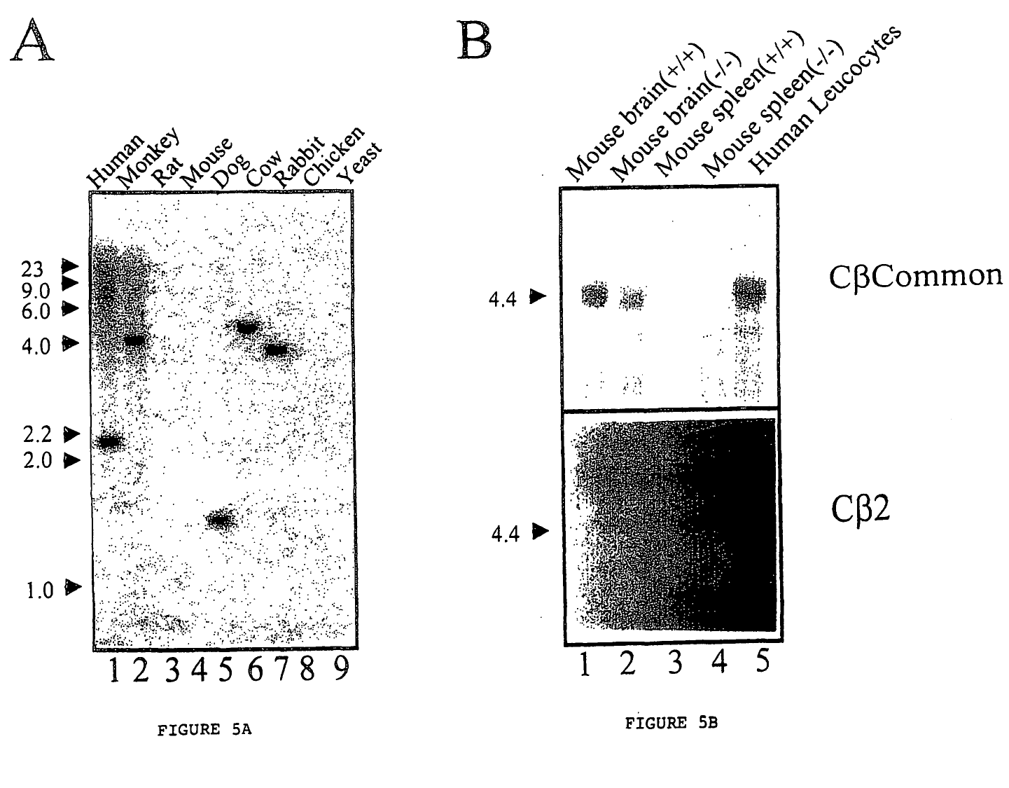

Previously we have identified three splice variants of Cβ in the mouse, Cβ1, Cβ2 and Cβ3 [14]. Based on the present work, it is apparent that mouse Cβ2 is not homologous to either bovine or the human Cβ2. Instead, mouse Cβ2 is homologous to what we now have designated human Cβ4. Thus, we investigated whether a Cβ splice variant similar to human Cβ2 was present in the mouse genome. A Zoo-blot containing genomic DNA isolated from human, monkey, rat, mouse, dog, cow, rabbit, chicken and yeast was hybridized using a DNA fragment corresponding to exon 1-2 of human Cβ. In FIG. 5 (panel A, lanes 1 to 9) we show that a DNA fragment was detected using Cβ2 specific probe in man, monkey, dog, cow, and rabbit. In contrast, the Cβ2 specific probe did not recognize any fragments in the rat and mouse suggesting that the Cβ2 specific exon is not present in the murine genome. To further substantiate this observation we isolated total RNA from human, wild type mice and mice that are ablated (knockout, KO) for exon 1A of the Cβ gene [16]. The RNA was isolated from immune tissues and brain since we observed high level expression of Cβ2 in human thymus, spleen and peripheral blood leukocytes and high level of the other Cβ splice variants in the brain (FIG. 4). The Northern blots were probed with a Cβ cDNA probe (expected to recognize all known Cβ splice variants) and a Cβ2 specific probe (see material and methods). In FIG. 5B (upper panel) we demonstrate that Cβ is present in the brain of wild type and Cβ exon 1 KO (lanes 1 and 2) and in human peripheral blood leukocytes (lane 5). The mouse spleen did not contain Cβ mRNA (lanes 3 and 4). When probing the same filter with the Cβ2 specific probe (FIG. 5, lower panel) Cβ2 message was only detected in human peripheral blood leukocytes (lane 5) whereas all the mouse tissues were negative for Cβ2 mRNA (lanes 1 to 4).

### REFERENCES

[1] Butcher, R. W., Ho, R. J., Meng, H. C., & Sutherland, E. W. (1965) Adenosine 3′,5′-monophosphate in biological materials. II. The measurement of adenosine 3′,5′-monophosphate in tissues and the role of the cyclic nucleotide in the lipolytic response of fat to epinephrine. J. Biol. Chem., 240, 4515-4523.

[2] Doskeland, S. O., Maronde, E., & Gjertsen, B. T. (1993) The genetic subtypes of cAMP-dependent protein kinase—functionally different or redundant? Biochim. Biophys. Acta, 1178, 249-258.

[3] Skalhegg, B. S. & Tasken, K. (1997) SPECIFICITY IN THE cAMP/PKA SIGNALING PATHWAY. DIFFERENTIAL EXPRESSION, REGULATION, AND SUBCELLULAR LOCALIZATION OF SUBUNITS OF PKA. Front Biosci., 2, d331-d342.

[4] Reinton, N., Haugen, T. B., Orstavik, S., Skalhegg, B. S., Hansson, V., Iahnsen, T., & Tasken, K. (1998) The gene encoding the C gamma catalytic subunit of cAMP-dependent protein kinase is a transcribed retroposon. Genomics, 49, 290-297.

[5] Beebe, S. J., Oyen, O., Sandberg, M., Froysa, A., Hansson, V., & Jahnsen, T. (1990) Molecular cloning of a tissue-specific protein kinase (C gamma) from human testis—representing a third isoform for the catalytic subunit of cAMP-dependent protein kinase. Mol. Endocrinol., 4,465-475.

[6] Zimmermann, B., Chiorini, J. A, Ma, Y., Kotin, R. M., & Herberg, F. W. (1999) PrKX is a novel catalytic subunit of the cAMP-dependent protein kinase regulated by the regulatory subunit type I. J. Biol. Chem., 274, 5370-5378.

[7] Showers, M. O. & Maurer, R. A. (1988) Cloning of cDNA for the catalytic subunit of cAMP-dependent protein kinase. Methods Enzymol., 159, 311-318.

[8] Thomis, D. C., Floyd-Smith, G., & Samuel, C. E. (1992) Mechanism of interferon action. cDNA structure and regulation of a novel splice site variant of the catalytic subunit of human protein kinase A from interferon-treated human cells. J. Biol. Chem., 267, 10723-10728.

[9] San Agustin, J. T., Leszyk, J. D., Nuwaysir, L. M., & Witman, G. B. (1998) The catalytic subunit of the cAMP-dependent protein kinase of ovine sperm flagella has a unique amino-terminal sequence. J. Biol. Chem., 273, 24874-24883.

[10]. Desseyn, J. L., Burton, K. A., & McKnight, G. S. (2000) Expression of a nonmyristylated variant of the catalytic subunit of protein kinase A during male germ-cell development. Proc. Natl. Acad. Sci. U.S.A., 97, 6433-6438.

[11] Reinton, N., Orstavik, S., Haugen, T. B., Jahnsen, T., Tasken, K., & Skalhegg, B. S. (2000) A novel isoform of human cyclic 3′,5′-adenosine monophosphate-dependent protein kinase, calpha-s, localizes to sperm midpiece. Biol. Reprod., 63, 607-611.

[12] Uhler, M. D., Chrivia, J. C., & McKnight, G. S. (1986) Evidence for a second isoform of the catalytic subunit of cAMP-dependent protein kinase [published erratum appears in J Biol Chem 1987 Apr. 15;262(11):5431]. J. Biol. Chem., 261,15360-15363.

[13] Wiemann, S., Kinzel, V., & Pyerin, W. (1991) Isoform C beta 2, an unusual form of the bovine catalytic subunit of cAMP-dependent protein kinase. J. Biol. Chem., 266, 5140-5146.

[14] Guthrie, C. R., Skalhegg, B. S., & McKnight, G. S. (1997) Two novel brain-specific splice variants of the murine Cbeta gene of cAMP-dependent protein kinase. J. Biol. Chem., 272, 29560-29565.

[15] Altschul, S. F., Madden, T. L., Schaffer, A. A., Zhang, J., Zhang, Z., Miller, W., & Lipman, D. J. (1997) Gapped BLAST and PSI-BLAST: a new generation of protein database search programs. Nucleic Acids Res., 25, 3389-3402.

[16] Qi, M., Zhuo, M., Skalhegg, B. S., Brandon, E. P., Kandel, E. R., McKnight, G. S., & Idzerda, R. L. (1996) Impaired hippocampal plasticity in mice lacking the Cbeta1 catalytic subunit of cAMP-dependent protein kinase. Proc. Natl. Acad. Sci. U.S.A., 93, 1571-1576.

[17] Herberg, F. W., Zimmermann, B., McGlone, M., & Taylor, S. S. (1997) Importance of the A-helix of the catalytic subunit of cAMP-dependent protein kinase for stability and for orienting subdomains at the cleft interface. Protein Sci., 6, 569-579.

[18] Yonemoto, W., McGlone, M. L., & Taylor, S. S. (1993) N-myristylation of the catalytic subunit of cAMP-dependent protein kinase conveys structural stability. J. Biol. Chem., 268, 2348-2352.

[19] Clegg, C. H., Ran, W., Uhler, M. D., & McKnight, G. S. (1989) A mutation in the catalytic subunit of protein kinase A prevents myristylation but does not inhibit biological activity. J. Biol. Chem., 264, 20140-20146.

[20] Carr, S. A, Biemann, K., Shoji, S., Parmelee, D. C., & Titani, S. (1982) n-Tetradecanoyl is the NH2-terminal blocking group of the catalytic subunit of cyclic AMP-dependent protein kinase from bovine cardiac muscle. Proc. Natl. Acad. Sci. U.S.A., 79, 6128-6131.

[21] Jedrzejewski, P. T., Girod, A., Tholey, A., Konig, N., Thullner, S., Kinzel, V., & Bossemeyer, D. (1998) A conserved deamidation site at Asn 2 in the catalytic subunit of mammalian cAMP-dependent protein kinase detected by capillary LC-MS and tandem mass spectrometry. Protein Sci., 7, 457-469.

[22] Zheng, J., Knighton, D. R., Xuong, N. H., Taylor, S. S., Sowadski, J. M., & Ten Eyck, L. F. (1993) Crystal structures of the myristylated catalytic subunit of cAMP-dependent protein kinase reveal open and closed conformations. Protein Sci., 2, 1559-1573.

[23] Yonemoto, W., McGlone, M. L., Grant, B., &. Taylor, S. S. (1997) Autophosphorylation of the catalytic subunit of cAMP-dependent protein kinase in Escherichia coli. Protein Eng, 10, 915-925:

[24] San Agustin, J. T. & Witman, G. B. (1994) Role of cAMP in the reactivation of demembranated ram spermatozoa. Cell Motil. Cytoskeleton, 27, 206-218.

[25] Gangal, M., Clifford, T., Deich, J., Cheng, X., Taylor, S. S., & Johnson, D. A. (1999) Mobilization of the A-kinase N-myristate through an isoform-specific intermolecular switch. Proc. Natl. Acad. Sci. U.S.A., 96, 12394-12399.

[26] Uhler, M. D., Carmichael, D. F., Lee, D. C., Chrivia, J. C., Krebs, E. G., & McKnight, G. S. (1986) Isolation of cDNA clones coding for the catalytic subunit of mouse cAMP-dependent protein kinase. Proc. Natl. Acad. Sci. U.S.A., 83, 1300-1304.

1. The genomic DNA sequence encoding novel human catalytic subunits Cβ2, Cβ4ab and Cβ4abc variants of c-AMP dependent protein kinase termed Cβ3, comprising the nucleotide sequence of SEQ ID NO:1.

2. The cDNA sequence encoding novel human catalytic subunits Cβ2, Cβ4ab and Cβ4abc variants of cAMP dependent protein kinase termed Cβ, comprising the nucleotide sequences of SEQ ID NOs: 2, 5 and 6, respectively.

3. The vectors comprising the DNA sequences according to claims 1 or 2.

4. The specific amino acid sequences of SEQ ID NOs 7, 8 and 9 of Cβ2, Cβ4ab and Cβ4abc, respectively.

5. A protein encoded by the nucleotide sequences according to claims 1 or 2.

6. A protein encoded by the specific DNA sequences according to claims 1 or 2 comprising the specific amino acid sequence of SEQ ID NOs: 7, 8 and 9.

7. A kit comprising Cβ2 specific DNA probes of claims 1 or 2.

8. The use of the Cβ2, Cβ4ab and Cβ4abc proteins of claims 1-6, for the preparation of pharmaceuticals.

9. The use of the Cβ2 protein of claims 1-6, for the preparation of a medicament for inhibition of the enzymatic activity of Cβ2.

10. The use of the Cβ2 protein of claims 1-6, for the preparation of a medicament that will specifically and irreversibly block Cβ2 interaction.

11. The use of the Cβ2 protein of claims 1-6, for the preparation of a medicament that will activate the enzymatic activity of Cβ2.

12. The use of the DNA sequences which is complementary to the Cβ2, Cβ4ab and Cβ4abc DNA according to claims 1 or 2 for the preparation of an anti sense drug.

13. A method for inspection and screening of patient T cells for the presence and location of the Cβ2 of claims 1-6, comprising:
a) collecting and washing in buffer of isolated peripheral blood T lymphocytes;
b) preparing for identification of Cβ2 protein by immunofluorescence, T cells are let to settle onto poly L-lysine coated cover slips following detergent-dependent lysis;
c) incubating with primary antibody (Ab), either irrelevant Ab or Cβ2 specific Ab, Ab overshoot will be removed by washing buffer and T cells incubated with secondary anti-IgG Ab conjugated with a fluorescent; and
d) inspection of T cells under fluorescent microscopy.

14. A method of screening patient T cells for membrane associated of the Cβ2 of claims 1-6, catalytic activity comprising:
a) collecting and washing in buffer of isolated peripheral blood T lymphocytes;
b) preparing of T cells by lysing in detergent buffer;
c) monitoring Cβ2 specific catalytic activity by established assay, Cβ1 activity is used as an internal control to determine relative activity.

15. A method for screening of patients for mutations in the Cβ2 gene of claims 1 or 2 and mRNA comprising:
a) collecting and washing in buffer of isolated peripheral blood T lymphocytes;
b) isolating of total RNA and genomic DNA according to established methods followed by RT-PCR using Cβ2 specific primers according to cDNA sequence of Cβ2 specific nucleotides or the Cβ2 specific exon, designated exon 1-2.

16. A product produced by the method according to claims 13, 14 and 15.

17. A test system for screening for inhibitory- or activating molecules of the Cβ2 protein of claims 1-6.

18. The product from the screening method according to claim 17.
